(19) United States
(12) Patent Application Publication
(10) Pub. No.: US 2010/0160332 A1
(43) Pub. Date: Jun. 24, 2010

(54) a2-Adrenoceptor Ligands

(75) Inventors: Maria Isabel Rozas Hernando, Dublin (IE); Fernando Rodriguez Royo, Logrono (ES)

Correspondence Address: SCULLY SCOTT MURPHY & PRESSER, PC, 400 GARDEN CITY PLAZA, SUITE 300, GARDEN CITY, NY, 11530, US

(73) Assignee: The Provost, Fellows and Scholars of the College of the Holy and Undivided Trinity of Queen Elizabe, Dublin (IE)

(21) Appl. No.: 12/644,668

(22) Filed: Dec. 22, 2009

(30) Foreign Application Priority Data
Dec. 23, 2008 (GB) 0823420.5

(51) Int. Cl. C07D 233/50; A61K 31/4168; A61K 31/498; C07D 241/36; C07C 257/00; A61K 31/155; C07D 405/02; A61K 31/4178; A61P 25/24; A61P 9/12; A61P 27/06

(52) U.S. Cl. 514/249; 548/333.1; 514/401; 544/353; 564/226; 514/632; 548/311.7; 514/402

(57) ABSTRACT

The adrenergic receptors or adrenoceptors are a family of G-protein coupled receptors split into α and β subclasses. The adrenoceptors have important roles in regulating a myriad of physiological conditions and their malfunction has been implicated in the pathophysiology of a number of diseases. Disclosed herein are a series of novel compounds which are ligands of the alpha2-adrenoceptor (α₂-ARs) subclass of adrenergic receptors. The invention also provides for pharmaceutical compositions comprising the novel compounds. The compounds are suitable for use in the manufacture of medicaments for the treatment of α₂-ARs associated disorders, such as depression and schizophrenia.

### FIELD OF THE INVENTION

A series of ligands of the alpha2-adrenoceptor (α2-AR) subclass of adrenergic receptors are disclosed herein. Such compounds find use as in the treatment of α2-AR associated disorders such as neurological conditions. The compounds disclosed herein are suitable for the treatment of depressive conditions and other α2-ARs associated disorders.

### BACKGROUND TO THE INVENTION

The adrenergic receptors or adrenoceptors are a family of G-protein coupled receptors split into α and β subclasses. The adrenoceptors have important roles in regulating a myriad of physiological conditions and their malfunction has been implicated in the pathophysiology of a number of diseases.

α-Adrenoceptors are further subdivided into α₁ and α₂ subclasses. The β₂ subclass of adrenoceptor can be found presynaptically, for example at nerve terminals, and postsynaptically, for example in vascular smooth muscle. Activation of presynaptic α₂ adrenoceptors inhibits noradrenaline release. Thus, antagonism of these receptors can be utilised to increase local concentrations of noradrenaline in nerve terminals.

Depression is a common mental disorder presenting symptoms including depressed mood, loss of interest or pleasure, feelings of guilt or low self-worth, disturbed sleep or appetite, low energy, and poor concentration. This condition affects people of any age and sex and it has been predicted that, by 2020, depression will be the second largest health burden following only heart diseases. Even though the pathophysiological origin of this disease continues to be unknown, the monoamine theory is the most widely accepted, i.e. depression is a result of a deficiency of brain monoamine (norepinephrine/noradrenaline [NA] or serotonin) activity.

Extensive research has been performed in the area of serotonin receptors, but investigations centred in the noradrenergic system remain less explored. In particular, it is well known that central noradrenergic transmission is regulated by inhibitory α₂-ARs, which are expressed on both somatodendritic areas and axon terminals. Hence, the activation of these α₂-ARs induces inhibition of NA release in the brain, and thus, it has been proposed that depression is associated with a selective increase in the high-affinity conformation of the α₂-ARs in the human brain. This enhanced α₂-AR activity could be implicated in decreased noradrenergic transmission described in the aetiology of depression. Thus, chronic treatment with antidepressants induces an in-vivo desensitization of the α₂-ARs regulating the local release of NA. Thus, the development of selective α₂-adrenoceptor antagonists can be considered as a new and effective therapeutical approach to the treatment of depressive disorders. It has been demonstrated that the administration of different α₂-AR antagonists both locally in the locus coeruleus or systemically increases the release of NA in the prefrontal cortex. Moreover, α₂-AR antagonists are also able to enhance the increase of NA induced by selective reuptake inhibitor antidepressant drugs.

Prior art antidepressants developed to target the adrenergic nervous system include Mianserin (1′) and Mirtazapine (2′) (infra), which show effective antidepressant activity by blockage of α₂-ARs.

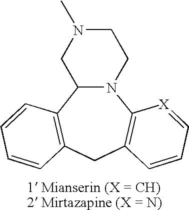

The success of these drugs supports α₂-AR targeting as a promising approach for the development of new therapeutics to treat depression.

The present inventors have disclosed a series of (bis)guanidine and (bis)2-aminoimidazoline derivatives as potential α₂-AR antagonists for the treatment of depression and other neurological conditions such as schizophrenia. Rozas at al. (Bioorg. Med. Chem. 2000, 8, 1567-1577 and Bioorg. Med. Chem. 2002, 10, 1525-1533) communicated the synthesis of a number of aromatic compounds bearing two guanidine or 2-aminoimidazoline groups at each end of a bis-phenyl chain (3′) (infra) and their α2-AR affinity, in human brain tissues (frontal cortex), was measured.

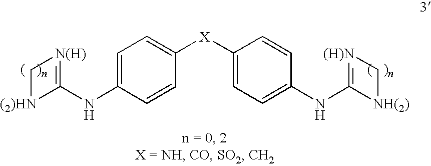

From this study, it was observed that compounds possessing 2-aminoimidazoline groups and the bis-aromatic motif observed in Mianserin and Mirtazapine, exhibited good α2-AR affinity, (pKi=8.80) and therefore, could be potential antidepressants.

Contrary to the above, further investigations by Rozas at al. (J. Med. Chem. 2007, 50, 4516-4527 and J. Med. Chem. 2008, 51, 3304-3312) produced a select few molecules absent a bis-aromatic motif (4′-7′) having an antagonist effect at α₂-ARs. These studies pointed to the fact that of the compounds synthesised the affinity of the guanidine containing substrates for the α₂-ARs is lower than that of the corresponding 2-aminoimidazoline analogues, intimating that 2-aminoimidazoline analogues are preferred substrates of the α₂-ARs.

The studies also showed an overwhelming preponderance for a 2-aminoimidazoline group over a guanidine moiety in compounds possessing α₂-AR antagonist activity (see 4′, 5′ and 7′ infra). Nitrogen based substitutions on the phenyl rings of these compounds illustrated a predilection for full valence substitution of the heteroatom in order to afford an antagonistic effect, e.g. dimethyl aniline derivative (4′). Mono-substitution of the phenyl ring with methyl ethers and methyl thioethers, i.e. oxygen and sulfur heteroatoms, invariably resulted in α₂-AR agonists. However, bicyclic dioxolane derivative (5′) proved to be the only oxygen substituted derivative showing antagonist behaviour at the α₂-AR. Of all the compounds disclosed in the aforementioned studies, compound 6′ was the only guanidine based compound depicting antagonist behaviour, and would strongly suggest that non-heteroatom substitution is a preference for guanidine based α₂-AR antagonists.

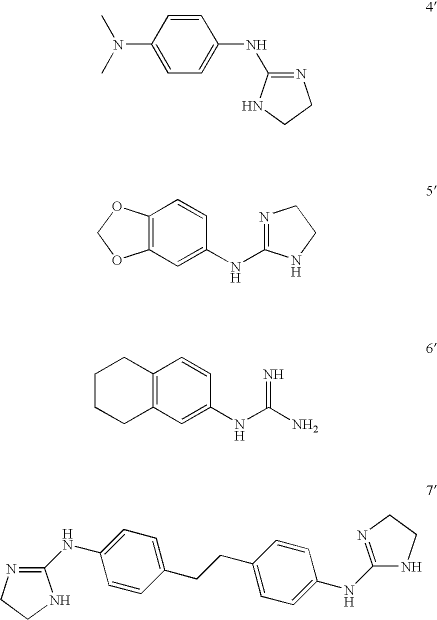

Thus, notwithstanding the state of the art it would still be desirable to provide novel compounds capable of exhibiting antagonistic behaviour at α₂-ARs which may find utility in the treatment of neurological disorders such as depression and schizophrenia.

### SUMMARY OF THE INVENTION

The present invention provides for a series of compounds that are ligands of the alpha2-adrenoceptor. In particular a series of compounds which are agonists and antagonists of the alpha2-adrenoceptor are disclosed herein. Such compounds may find utility in the manufacture of medicaments for the treatment of alpha2-adrenoceptor associated disorders.

Compounds herein defined as antagonists may find utility in the manufacture of medicaments for the treatment of alpha2-adrenoceptor associated disorders. In particular, the alpha2-adrenoceptor antagonists of the present invention may find utility in the treatment of depression and schizophrenia. Antagonist compounds according to the present invention may enhance synaptic levels of noradrenaline in the brain. Such antagonist compounds according to the present invention may increase synaptic levels of noradrenaline in the brain by local or systemic administration.

Similarly, compounds herein defined as agonists may find utility in the manufacture of medicaments for the treatment of alpha2-adrenoceptor associated disorders. In particular, the alpha2-adrenoceptor agonists of the present invention may find utility in analgesia and the treatment of glaucoma and hypertension. Agonist compounds according to the present invention may act on postsynaptic alpha2-adrenoceptors, for example in vascular smooth muscle. Alternatively, agonist compounds according to the present invention may inhibit excitatory neurotransmission in the sympathetic nervous system resulting in sedation or analgesia.

Compounds of the present invention may be used as new and effective therapeutics or in the manufacture of such therapeutics for use in the treatment of alpha2-adrenoceptor associated disorders such as depression, schizophrenia, glaucoma and analgesia.

In one aspect the present invention provides for a compound comprising,

an aromatic ring, optionally substituted with at least one C₁-C₅alkyl, selected from the group comprising benzene, thiophene, pyrrole, furan, oxazole, thiazole, pyrazole, pyridine, pyrimidine, pyridazine, and pyrazine;

a single heteroatom, selected from O, N or S, covalently bonded to the aromatic ring, wherein the heteroatom is monoalkylated with a C₁-C₅ alkyl or a C₁-C₅ alkyl chain substituted with at least one of a halogen, hydroxy, thiol, or amine; and

a guanidine, a hydroxyguanidine, 2-aminoimidazole, amidine or isourea moiety optionally substituted with at least one of OH, N-tert-butoxycarbonate, or C₁-C₅ alkyl, covalently bonded:
a. to the aromatic ring; or
b. to a C₁-C₅ alkyl or a C₁-C₅ alkyl chain substituted with at least one of a halogen, hydroxy, thiol, or amine, wherein the C₁-C₅ alkyl is covalently bonded to the aromatic ring;

a tautomer thereof, a pharmaceutically acceptable salt thereof, or a hydrate thereof,

with the proviso that when the aromatic ring is benzene having a guanidine or 2-aminoimidazole moiety covalently bound thereto, and the heteroatom is O or S, the heteroatom it is not alkylated with a methyl group.

The aromatic ring may be selected from the group comprising benzene, thiophene, pyrimidine or pyridine. Desirably, the aromatic ring is benzene or thiophene. Further desirably, the aromatic ring is benzene. The heteroatom may be N. Desirably, the N heteroatom is substituted with a C₂-C₅ alkyl.

The guanidine, hydroxyguanidine, isourea, amidine or 2-aminoimidazole moiety may be covalently bonded to a C₁-C₅ alkyl, wherein the C₁-C₅ alkyl is covalently bonded to the aromatic ring. Desirably, the guanidine, hydroxyguanidine, isourea, amidine or 2-aminoimidazole moiety may is covalently bonded to the aromatic ring. The guanidine, a hydroxyguanidine, or isourea may be covalently bonded to the aromatic ring. The guanidine or hydroxyguanidine moiety may be covalently bonded to the aromatic ring.

The guanidine, hydroxyguanidine, isourea, amidine or 2-aminoimidazole moiety may be substituted with at least one of OH, or C₁-C₅ alkyl. The guanidine, hydroxyguanidine, isourea, amidine or 2-aminoimidazole moiety may be substituted with at least one OH. Desirably, the guanidine, hydroxyguanidine, isourea, amidine or 2-aminoimidazole moiety is not substituted and the valence on the heteroatoms is fulfilled with H.

In a further aspect the present invention relates to a compound of the general formula (I):

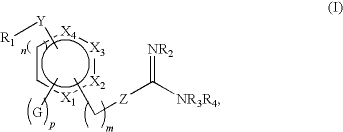

wherein n is 0 or 1;
m is 0 to 5;
p is 0,1 or 2
G is C₁-C₅ alkyl, wherein when p is 2, G₁ and G₂ can be the same or different C₁-C₅ alkyl groups;
Y is N, O or S;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
   Z is O, C or N—R₅;
   R₅ is H or C₁-C₅ alkyl;

X₁ to X₄ are the same or different and are selected from the group comprising C, N, S and O;

wherein when n is O, X₄ is C;
X₁ is S, O, or N; and
X₂ and X₃ are C or N, with the proviso that X₂ is C when X₁ is S or O; and

further provided that when n is 1, X₁ to X₄ are C or N, such that at least two of X₁ to X₄ are always C;
with the proviso that when X₁ to X₄ are C, n is 1, m is 0, Z and R₂ to R₄ (i.e. the moiety comprising Z, C, NR₂ and NR₃R₄) together define a guanidine or 2-aminoimidazole moiety and Y is O or S, R₁ is not methyl,

a tautomer thereof, a pharmaceutically acceptable salt thereof, or a hydrate thereof.

As used herein the term “tautomer” is with reference to the guanidine, hydroxyguanidine, isourea, etc. moiety embraced by the structure (A) capable of facile proton transfer between the heteroatoms. The example provided adjacent structure (A) is a clarifying example only, and is by no means to be considered a limitation of what Z, R₂, R₃ and R₄ must embrace in order for tautomerism to occur.

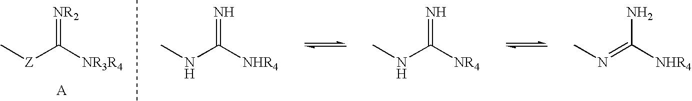

As used herein, the term “C₁-C₅ alkyl” embraces branched and straight chain C₁-C₅ alkyl.

Desirably, when n is 1 the substituent comprising the variable Y and the substituent comprising the variable Z are in a 1,4-relationship on the aromatic ring. Further desirably, when n is 0 or 1, the combination of substituents X₁ to X₄ will result in an aromatic ring, i.e. the heteroatom substituents will not break aromaticity in the ring.

R₂ to R4 may be the same or different and may be selected from the group comprising H, OH, N-tert-butoxycarbonate, or C₁-C₅ alkyl. R₁ may be C₁-C₅ alkyl. m may be 0. p may be 0. Desirably, m is 0, p is 0 and R₁ is C₁-C₅ alkyl. Further desirably, m is 0, p is 0, R₁ is C₁-C₅ alkyl and Z is N—R₅. Further desirably, m is 0, p is 0, R₁ is C₁-C₅ alkyl, and Z is N—H. For example, m is 0, p is 0, R₁ is C₁-C₅ alkyl, Z is N—H and R₂ to R₄ are the same or different and are selected from the group comprising H, OH, N-tert-butoxycarbonate, or C₁-C₅ alkyl.

In one embodiment the present invention provides for a compound of the general formula (ID:

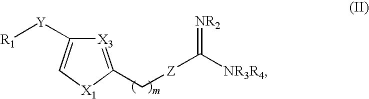

wherein m is 0 to 5;
Y is N, O or S;
Ris a C₁-C₅ alkyl or a C₁-C₅ alkyl chain substituted with at least one of a halogen, hydroxy, thiol, or amine;

R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
X₁ is N, S or O; and
X₃ is C or N,

a tautomer thereof, a pharmaceutically acceptable salt thereof, or a hydrate thereof.

X₃ may be C. R₂ to R₄ may be the same or different and may be selected from the group comprising H, OH, N-tert-butoxycarbonate, or C₁-C₅ alkyl. R₁ may be C₁-C₅ alkyl. m may be 0, X₁ may be S. Desirably, m is 0, X₁ is 5, X₃ is C and R₁ is C₁-C₅ alkyl. Further desirably, m is 0, X₁ is 5, X₃ is C, R₁ is C₁-C₅ alkyl and Z is N—R₅. For example, m is 0, X₁ is S, X₃ is C, R₁ is C₁-C₅ alkyl and Z is N—H. Such as, m is 0, X₁ is 5, X₃ is C, R₁ is C₁-C₅ alkyl, Z is N—H and R₂ to R₄ are the same or different and may be selected from the group comprising H, OH, N-tert-butoxycarbonate, or C₁-C₅ alkyl.

In a further embodiment the present invention provides for a compound of the general formula (III):

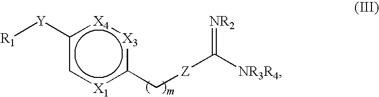

wherein m is 0 to 5;
Y is N, 0 or 5;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
X₁, X₃, and X₄ are C or N, such that at least one of X₁, X₃, and X₄ is always C;

with the proviso that when X₁, X₃, and X₄ are C, m is 0, Z and R₂ to R₄ (i.e. the moiety comprising Z, C, NR₂ and NR₃R₄) together define a guanidine or 2-aminoimidazole moiety and Y is O or 5, R₁ is not methyl,
a tautomer thereof, a pharmaceutically acceptable salt thereof, or a hydrate thereof.

R₂ to R₄ may be the same or different and may be selected from the group comprising H, OH, N-tert-butoxycarbonate, or C₁-C₅ alkyl. R₁ may be C₁-C₅ alkyl m may be 0. X₁ may be or N and X₃ and X₄ are C. Desirably, m is 0, X₁ is C or N, X₃ and X₄ are C and R₁ is C₁-C₅ alkyl. Further desirably, m is 0, X₁ is C or N, X₃ and X₄ are C, R₁ is C₁-C₅ alkyl and Z is N—R₅. For example, m is 0, X₁ is C or N, X₃ and X₄ are C, R₁ is C₁-C₅ alkyl and Z is N—H. Such as, m is 0, X₁ is C or N, X₃ and X₄ are C, R₁ is C₁-C₅ alkyl, Z is N—H and R₂ to R₄ are the same or different and may be selected from the group comprising H, OH, N-tert-butoxycarbonate, or C₁-C₅ alkyl.

In yet a further embodiment, the present invention provides for a compound of the general formula (IV):

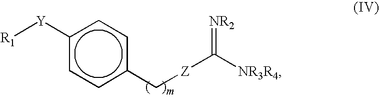

wherein m is 0 to 5;
Y is N, O or S;

R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;

with proviso that when m is 0, Z and R₂ to R₄ (i.e. the moiety comprising Z, C, NR₂ and NR₃R₄) together define a guanidine or 2-aminoimidazole moiety and Y is O or S, R₁ is not methyl,

a tautomer thereof, a pharmaceutically acceptable salt thereof, or a hydrate thereof.

R₂ to R4 may be the same or different and may be selected from the group comprising H, OH, N-tert-butoxycarbonate, or C₁-C₅ alkyl. R₁ may be C₂-C₅ alkyl. Z may be N—R₅. Desirably, R₁ is C₂-C₅ alkyl and Z is N—R₅. For example, R₁ is C₂-C₅ alkyl and Z is N—H. Such as, R₁ is C₂-C₅ alkyl, Z is N—H and R₂ to R₄ are the same or different and may be selected from the group comprising H, OH, N-tert-butoxycarbonate, or C₁-C₅ alkyl.

The compound of the present invention may be of the general formula (V):

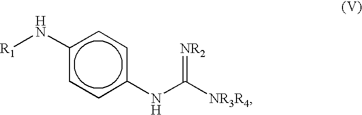

wherein R₁ is a C₂-C₅ alkyl or a C₂-C₅ alkyl substituted with at least one of a halogen, hydroxy, thiol, or amine; and

R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring,

a tautomer thereof, a pharmaceutically acceptable salt thereof, or a hydrate thereof.

R₂ to R₄ may be the same or different and may be selected from the group comprising H, OH, N-tert-butoxycarbonate, or C₁-C₅ alkyl. R₁ may be C₂-C₅ alkyl. Desirably, R₁ is C₂-C₅ alykl and R₂ to R₄ are the same or different and are selected from the group comprising H, OH, N-tert-butoxycarbonate, or C₂-C₅ alkyl. Further desirably, R₁ is a C₂-C₅ alkyl and R₂ to R₄ are H.

In one embodiment, the compound of the present invention is

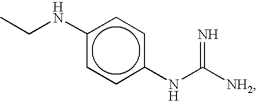

a tautomer thereof, a pharmaceutically acceptable salt thereof, or a hydrate thereof.

In a further aspect the present invention relates to a pharmaceutical composition comprising a compound according to the present invention, a tautomer thereof, a pharmaceutically acceptable salt thereof, or a hydrate thereof, together with a pharmaceutical acceptable carrier or excipient.

In one embodiment the pharmaceutical composition of the present invention comprises:

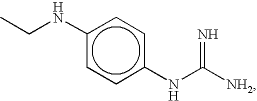

a tautomer thereof, pharmaceutically acceptable salt thereof, or a hydrate thereof, together with a pharmaceutical acceptable carrier or excipient.

The present invention also provides for a compound according to the present invention, a tautomer thereof, a pharmaceutically acceptable salt thereof, or a hydrate thereof, for the treatment of an alpha2-adrenoceptor associated disorder.

Desirably, the present invention provides for a compound of the structure,

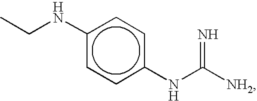

a tautomer thereof, a pharmaceutically acceptable salt thereof, or a hydrate thereof, for the treatment of an alpha2-adrenoceptor associated disorder.

The alpha2-adrenoceptor associated disorder may be at least one of depression or schizophrenia. Desirably, the alpha2-adrenoceptor associated disorder is depression.

The present invention also provides for use of a compound according to the present invention, a tautomer thereof, a pharmaceutically acceptable salt thereof, or a hydrate thereof, in the manufacture of a medicament for the treatment of an alpha2-adrenoceptor associated disorder.

Desirably, use of a compound according to the present invention comprises use of:

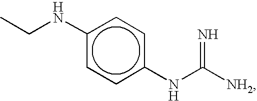

a tautomer thereof, a pharmaceutically acceptable salt thereof, or a hydrate thereof, in the manufacture of a medicament for the treatment of an alpha2-adrenoceptor associated disorder.

The alpha2-adrenoceptor associated disorder may be at least one of depression or schizophrenia. Desirably, the alpha2-adrenoceptor associated disorder is depression.

In a further aspect, the present invention provides that compounds according to the present invention are alpha2-adrenoceptor antagonists.

The invention further relates to a method of treating an alpha2-adrenoceptor associated disorder in a patient in need thereof, comprising administering to the patient a pharmaceutically effective amount of a compound according to the present invention, a tautomer thereof, a pharmaceutically acceptable salt thereof, or a hydrate thereof. The alpha2-adrenoceptor associated disorder may be selected from at least one of depression or schizophrenia. Desirably, the alpha2-adrenoceptor associated disorder is depression.

In yet a further aspect the present invention provides an alpha2-adrenoceptor agonist selected from the group comprising:

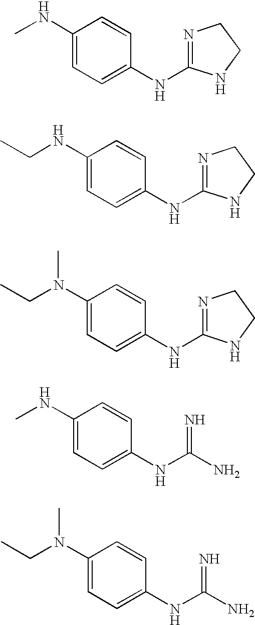

The alpha2-adrenoceptor agonists of the present invention may find utility in the treatment of at least one of analgesia, glaucoma or hypertension. The alpha2-adrenoceptor agonists of the present invention may further find utility in the manufacture of medicaments for analgesia or for the treatment of at least one of hypertension or glaucoma. Further desirably, the alpha2-adrenoceptor agonists of the present invention may find utility in the manufacture of medicaments for analgesia and the treatment of glaucoma.

The invention provides for a method of treating an alpha2-adrenoceptor associated disorder in a patient in need thereof, comprising administering to the patient a pharmaceutically effective amount of an alpha2-adrenoceptor agonist according to the present invention, a tautomer thereof, a pharmaceutically acceptable salt thereof, or a hydrate thereof. The alpha2-adrenoceptor associated disorder may be selected from at least one of analgesia, hypertension or glaucoma. Desirably, the alpha2-adrenoceptor associated disorder is glaucoma.

The present invention provides for a series of novel alpha2-adrenoceptor ligands. The ligands are readily synthesised from inexpensive commercially available starting materials as illustrated in the detailed description of the invention section. The relatively straightforward synthesis of these molecules and their resultant utility makes them desirable targets as medicaments for pharmacological intervention in alpha2-adrenoceptor implicated disease states.

Of particular note, the compounds all show desirable affinities towards the alpha2-adrenoceptor. As such alpha2-adrenoceptor ligands according to the present invention hold promise in the therapeutic intervention of at least one of depression, schizophrenia, glaucoma, hypertension or analgesia.

As described in the detailed description of the invention antagonist compounds of the present invention increase levels of noradrenaline in-vitro. Furthermore, antagonist compounds of the present invention increase levels of noradrenaline in-vivo. Notably, antagonist compounds according to the present invention have provided results comparable and superior to known potent alpha2-adrenoceptor antagonists in both in-vivo and in-vitro testing.

Where suitable, it will be appreciated that all optional and/or preferred features of one embodiment of the invention may be combined with optional and/or preferred features of another/other embodiment(s) of the invention.

### BRIEF DESCRIPTION OF THE DRAWINGS

Additional features and advantages of the present invention are described in, and will be apparent from, the detailed description of the invention and from the drawings in which:

FIG. 1 illustrates the synthetic methods utilised to prepare compounds according to the present invention;

FIG. 2 illustrates the effects of local administration of compound 8b according to the present invention (1-100 μM), RX821002 (1-100 μM) and artificial cerebrospinal fluid (aCSF) in the Pre-frontal Cortex (PFC);

FIG. 3 illustrates the effects of intraperitoneal administration of compound 8b and saline on extracellular NA levels evaluated in the PFC; and

FIG. 4 illustrates the rat tail suspension test for a number of compounds including compound 8b according to the present invention.

### DETAILED DESCRIPTION OF THE INVENTION

### Compound Synthesis

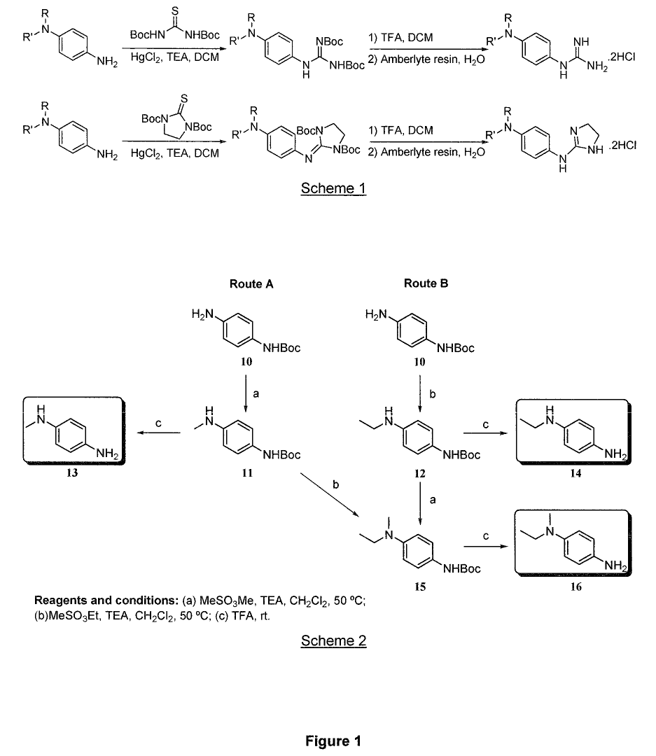

Reaction of primary aromatic amines with one equivalent of either N,N′-bis(tert-butoxycarbonyl)thiourea (guanidine precursor) or N,N′-di(tert-butoxy carbonyl)imidazoline-2-thione (2-aminoimidazoline precursor) in the presence of mercury (II) chloride and an excess of triethylamine as shown in Scheme 1 FIG. 1 was the chosen synthetic route to the compounds of the present invention.

In all cases, the reaction was carried out in dichloromethane and the BOC-protected precursors obtained in the first step of the synthesis were purified by a quick neutral alumina flash column chromatography. Standard removal of the BOC groups with an excess of trifluoroacetic acid in dichloromethane followed by treatment with Amberlyte resin in water led to the hydrochloride salts of the target molecules in overall good yields ranging from 48% to 75%. The structures and yields of the compounds prepared are displayed in Table 1.

TABLE 1
Overall, first and second stage yields (in %) obtained for the compounds prepared.

| | | 1^{st} | | | 2^{nd} | |
|---|---|---|---|---|---|---|
| Compd | Structure | Stage | Compd | Structure | Stage | Overall |
| 4a | 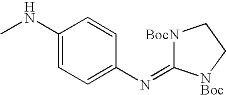 | 60 | 4b | 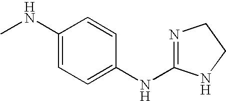 | 93 | 56 |
| 5a | 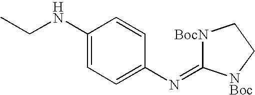 | 52 | 5b | 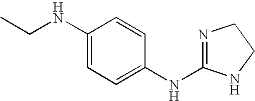 | 93 | 48 |
| 6a | 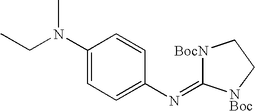 | 68 | 6b | 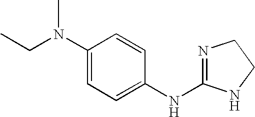 | 93 | 63 |
| 7a | 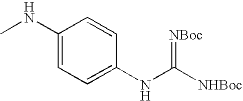 | 77 | 7b | 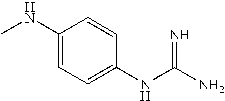 | 95 | 73 |
| 8a | 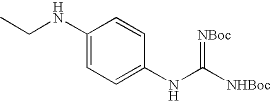 | 72 | 8b | 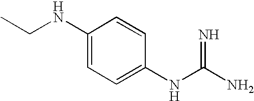 | 94 | 68 |
| 9a | 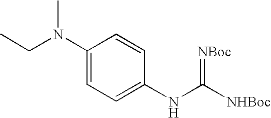 | 78 | 9b | 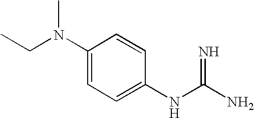 | 96 | 75 |

Compound 10 (N-BOC-p-phenylenediamine) was used, as an advanced intermediate, for the synthesis of the final compounds 4b-9b as depicted in Scheme 2 FIG. 1. Thus, treatment of derivative 10 with one equivalent of methyl methanesulfonate and triethylamine in dichloromethane led to the monomethylated derivative 11 in a 25% yield after column chromatography in silica gel. The ethylamine compound 12 was obtained in a similar manner via ethyl methanesulfonate. In this case, the yield increased to 34%. In both reactions, formation of dialkylated products was observed.

All the chemicals used for the synthesis described in Schemes 1 and 2 of FIG. 1 are commercially available either from Aldrich or Fluka. The BOC-protected amines 12 and 15 are new, whereas the methylamine derivative 11 has been previously described in the literature, as the anilines 13, 14 and 16.

General Procedure for the Synthesis of BOC-Protected 2-iminoimidazolidine and BOC-Protected Guanidine Derivatives: Method A.

Each of the corresponding anilines was treated in DCM at 0° C. with 1.1 equivalents of mercury (II) chloride, 1.0 equivalent of N,N′-di(tert-butoxycarbonyl)imidazolidine-2-thione (for the 2-aminoimidazoline precursors) or N,N′-di(tert-butoxycarbonyl)thiourea (for the guanidine precursors) and 3.1 equivalents of TEA. The resulting mixture was stirred at 0° C. for 1 hour and for the appropriate duration at room temperature. Then, the reaction mixture was diluted with EtOAc and filtered through a pad of Celite to get rid of the mercury sulfide formed. The filter cake was rinsed with EtOAc. The organic phase was washed first with water (2×30 mL), then with brine (1×30 mL), dried over anhydrous Na₂SO₄ and concentrated under vacuum to give a residue that was purified by neutral alumina column flash chromatography, eluting with the appropriate hexane:EtOAc mixture.

### General Procedure for the Synthesis of the Dihydrochloride Salts: Method B.

Each of the corresponding BOC-protected precursors (0.5 mmol) was treated with 15 mL of a 50% solution of trifluoroacetic acid in DCM for 3 h. After that time, the solvent was eliminated under vacuum to generate the trifluoroacetate salt. This salt was dissolved in 20 mL of water and treated for 24 h with IRA400 Amberlyte resin in its Cl⁻ form. Then, the resin was removed by filtration and the aqueous solution washed with DCM (2×10 mL). Evaporation of the water afforded the pure dihydrochloride salt. Absence of the trifluoroacetate salt was checked by ¹⁹F NMR.

### General Procedure for the Alkylation of Primary and Secondary Amines: Method C.

The alkylating agent (10.0 mmol of methyl methanesulfonate or ethyl methanesulfonate) and 10.0 mmol of TEA were added at 0° C. over a solution containing 10.0 mmol of the corresponding amine in DCM (12 mL). The resulting mixture was heated at reflux temperature for 15 h and after cooling it was diluted with 40 mL of DCM, washed with a 10% NaOH solution (2×15mL) and water (2×15 mL). The organic phase was dried over anhydrous Na₂SO₄, filtered and concentrated under vacuum to give a residue that was purified by silica gel column chromatography, eluting with the appropriate hexane:EtOAc mixture.

### General Procedure for the BOC-Deprotection and Preparation of the Starting Material Amines: Method D.

A solution containing 10.0 mmol of the BOC-protected compound (11, 12 or 15) in 15 mL of TFA was stirred at room temperature for 2 h. Then, the solvent was eliminated under vacuum to generate the trifluoroacetate salt. This salt was redissolved in 20 mL of an aqueous solution of NaOH (2M) and washed with DCM (3×15 mL). The organic layer was washed with water (2×10 mL), dried over anhydrous Na₂SO₄, filtered and concentrated to give the corresponding free amine as an oil.

### Dihydrochloride Salt of N-imidazolidin-2-ylidene-N′-methyl-benzene-1,4-diamine (4b): Method B

Red solid (93%); mp 230-232° C.; ¹H NMR (D₂O) δ 3.07 (s, 3H, CH₃), 3.74 (s, 4H, CH₂), 7.44 (d, 2H, J=8.5 Hz, Ar.), 7.54 (d, 2H, J=8.5 Hz, Ar.); ¹³C NMR (D₂O) δ 36.4 (CH₃), 42.3 (CH₂), 123.1, 125.2, 134.1, 136.0 (Ar.), 158.0 (CN); HRMS (ESI⁺) m/z calcd. [M+H]⁺ 191.1291, found 191.1291. Anal. Calcd. for (C₁₀H₁₆Cl₂N₄.0.4H₂O): C, H, N.

### Dihydrochloride Salt of N-ethyl-N′-imidazolidin-2-ylidene-benzene-1,4-diamine (5b): Method B

White solid (93%); mp 198-200° C.; ¹H NMR (D₂O) δ 1.29 (t, 3H, J=7.5 Hz, CH₃CH₂), 3.47 (q, 2H, J=7.5 Hz, CH₃CH₂), 3.75 (s, 4H, CH₂), 7.45 (d, 2H, J=9.0 Hz, Ar.), 7.53 (d, 2H, J=9.0 Hz, Ar.); ¹³C NMR (D₂O) δ 9.7(CH₃CH₂, 42.3 (CH₂), 46.9(CH₃CH₂, 123.7, 125.1, 132.4, 136.0 (Ar.), 158.0 (CN); HRMS (ESI⁺) m/z calcd. [M+H]⁺ 205.1448, found 205.1443. Anal. Calcd. for (C₁₁H₁₆Cl₂N₄.1.5H₂O): C, H, N.

### fDihydrochloride Salt of N-Ethyl-N′-imidazolidin-2-ylidene-N-methyl-benzene-1,4-diamine (6b): Method B

White solid (93%); mp 48-50° C.; ¹H NMR (D₂O) δ 1.15 (t, 3H, J=7.5 Hz, CH₃CH₂), 3.26 (s, 3H, CH₃), 3.63 (q, 2H, J=7.5 Hz, CH₃CH₂), 3.76 (s, 4H, CH₂), 7.49 (d, 2H, J=9.0 Hz, Ar.), 7.64 (d, 2H, J=9.0 Hz, Ar.); ¹³C NMR (D₂O) δ 9.3 (CH₃CH₂, 42.3 (CH₂), 44.1 (CH₃), 54.8 (CH₃CH₂, 122.5, 125.1, 136.6, 137.4 (Ar.), 157.9 (CN); HRMS (ESI⁺) m/z 219.1604 calcd. [M+H]⁺, found 219.1605. Anal. Calcd. for (C₁₂H₂₀Cl₂N₄.1.8H₂O): C, H, N.

### Dihydrochloride salt of N-(4-methylamino-phenyl)-guanidine (7b): Method B

Pinkish solid (95%); mp 74-76° C.; ¹H NMR (D₂O) δ 3.08 (s, 3H, CH₃), 7.46 (d, 2H, J=8.5 Hz, Ar.), 7.56 (d, 2H, J=9.0 Hz, Ar.); ¹³C NMR (D₂O) δ 36.4 (CH₃), 123.3, 126.7, 134.6, 135.3 (Ar.), 155.6 (CN); HRMS (ESI⁺) m/z 165.1135 calcd. [M+H]⁺, found 165.1138. Anal. Calcd. for (C₈H₁₄Cl₂N₄.1.3H₂O): C, H, N.

### Dihydrochloride salt of N-(4-ethylamino-phenyl)-guanidine (8b): Method B

White solid (94%); mp 126-128° C.; ¹H NMR (D₂O) δ 1.31 (t, 3H, J=7.0 Hz, CH₃CH₂), 3.48 (q, 2H, J=7.0 Hz, CH₃CH₂), 7.49 (d, 2H, J=8.0 Hz, Ar.), 7.54 (d, 2H, J=8.0 Hz, Ar.); ¹³C NMR (D₂O) δ 9.8(CH₃CH₂, 46.7(CH₃CH₂, 123.6, 126.7, 133.2, 135.0 (Ar.), 155.6 (CN); HRMS (ESI⁺) m/z 179.1297 calcd. [M+H]⁺, found 179.1303. Anal. Calcd. for (C₉H₁₆Cl₂N₄.1.0H₂O); C, H, N.

### Dihydrochloride salt of N-[4-(ethyl-methyl-amino)-phenyl]-guanidine (9b): Method B

White solid (96%); mp decomposes over 150° C.; ¹H NMR (D₂O) δ 1.16 (t, 3H, J=7.0 Hz, CH₃CH₂), 3.27 (s, 3H, CH₃), 3.64 (q, 2H, J=7.0 Hz, CH₃C H₂), 7.52 (d, 2H, J=8.5 Hz, Ar.), 7.65 (d, 2H, J=8.5 Hz, Ar.); ¹³C NMR (D₂O) δ 9.2 (CH₃CH₂, 44.1 (CH₃, 54.7 (CH₃C₂, 122.6, 126.6, 135.9, 137.7 (Ar.), 155.5 (CN); HRMS (ESI⁺) m/z 193.1448 calcd. [M+H]⁺, found 193.1450. Anal. Calcd. for (C₁₀H₁₈Cl₂N₄.0.2H₂O): C, H, N.

### N-Methyl-benzene-1,4-diamine (13): Method D

Red oil (87%); IR (nujol) 3400, 3339, 3222 cm⁻¹; ¹H NMR (CDCl₃) δ 2.76 (s, 3H, CH₃), 3.29-3.38 (m, 3H, NH₂+CH₃NH), 6.52 (d, 2H, J=9.0 Hz, Ar.), 6.62 (d, 2H, J=9.0 Hz, Ar.); ¹³C NMR (CDCl₃) δ 31.4(CH₃ 113.7, 116.5, 137.4, 142.2 (Ar.)

### N-Ethyl-benzene-1,4-diamine (14): Method D

Red oil (84%), IR (nujol) 3378, 3335, 3217 cm⁻¹; ¹H NMR (CDCl₃) δ 1.24 (t, 3H, J=7.0 Hz, CH₃CH₂), 2.85-3.19 (m, 5H, NH₂+NH+CH₃CH₂), 6.53 (d, 2H, J=8.5 Hz, Ar.), 6.62 (d, 2H, J=8.5 Hz, Ar.); ¹³C NMR (CDCl₃) δ 15.0(CH₃CH₂, 39.6(CH₃CH₂ 114.5, 116.8, 137.6, 141.6 (Ar.).

### N-Ethyl-N-methyl-benzene-1,4-diamine (16): Method D

Brownish oil (89%); IR (nujol) 3433, 3345 cm⁻¹; ¹H NMR (CDCl₃) δ 1.10 (t, 3H, J=7.0 Hz, CH₃CH₂), 2.82 (s, 3H, CH₃), 3.21-3.43 (m, 4H, NH₂+CH₃CH₂), 6.67 (d, 2H, J=9.0 Hz, Ar.), 6.71 (d, 2H, J=9.0 Hz, Ar.); ¹³C NMR (CDCl₃) δ 10.9 (CH₃CH₂, 38.4 (CH₃), 48.2(CH₃CH₂ 115.8, 116.5, 137.5, 143.1 (Ar.).

### Pharmacology Tests

The affinity of all compounds prepared towards the α₂-ARs in human brain PFC (pre-frontal cortex) tissue was measured by competition with the α₂-AR selective radioligand [³H]RX821002 (2-methoxy-idazoxan), which was used at a constant concentration of 1 nM. The affinities obtained, expressed as pKᵢ, are displayed in Table 2. Three of the most common α₂-AR ligands (Idazoxan, Clonidine and RX821002) were used as references.

TABLE 2
α₂-ARs affinity values (expressed as Kᵢ and pKᵢ)
obtained for all compounds studied.

| Com- | | | |
|---|---|---|---|
| pound | Structure | Kᵢ (±SEM) | pKᵢ^{§†} |
| RX821002 | 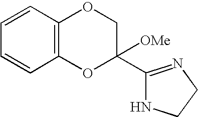 | 0.9 ± 0.2 | 9.04 |
| Idazoxan | 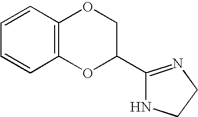 | 51 ± 9 | 7.29 |
| Clonidine | 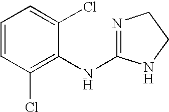 | 21 ± 9 | 7.68 |
| 1* | 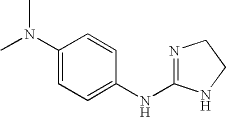 | 38 ± 7 | 7.42 |
| 2* | 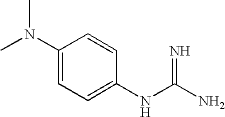 | 87 ± 17 | 7.06 |
| 3* | 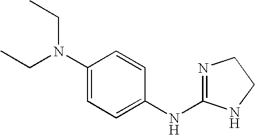 | 82 ± 44 | 7.09 |
| 17* | 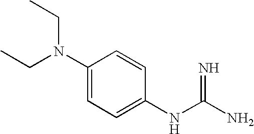 | 462 ± 201 | 6.34 |
| 4b | 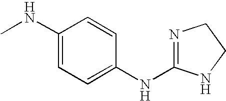 | 53 ± 7 | 7.27 |
| 5b | 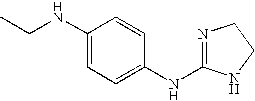 | 175 ± 36 | 6.75 |
| 6b | 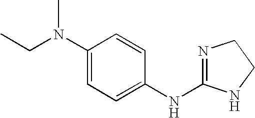 | 41 ± 8 | 7.38 |
| 7b | 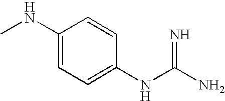 | 113 ± 16 | 6.95 |
| 8b | 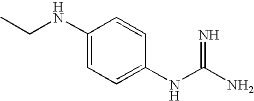 | 261 ± 35 | 6.58 |
| 9b | 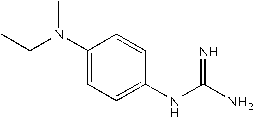 | 75 ± 11 | 7.12 |

*Compounds previously prepared by the inventors.
^{§}Affinity was measured by competition assays with the α₂-AR selective radioligand [³H]RX821002 (1 nM) in PFC human tissues.
^{†}Cortical membranes from human postmortem brains were incubated at 25° C. for 30 min with [³H]RX821002 (1 nM) in the absence or presence of the competing compounds (10⁻¹² M to 10⁻³ M, 10 concentrations).

In the 2-aminoimidazoline series, all new compounds displayed a pKᵢ value higher than 7 except for the ethylamino derivative 5b (pKᵢ=6.75, see Table 2). These values are within the range of the well-known α₂-AR ligands Idazoxan and/or Clonidine, with compound 1 keeping the highest affinity of the series (pKᵢ=7.42). Among the new derivatives, the pKᵢ value obtained for the analogue 6b is very similar to that of compound 1, whilst the secondary methylamino derivative 4b showed a slightly lower affinity.

With reference to the novel guanidine containing compounds, the order of affinity towards the α₂-ARs is the same as that for their 2-aminoimidazoline counterparts. Thus, the ethyl-methylamino compound 9b shows the highest pKᵢ (7.12, see Table 2) whereas the monoethylamino derivative 8b possesses the lowest affinity with a pKᵢ value of 6.58.

It is noteworthy that the affinity shown by compound 9b is the best of its series, and within the range of the α₂-AR antagonist Idazoxan. Five out of the six derivatives exhibited in Table 2 with a dialkyl amine in para (compounds 1, 2, 3, 6b and 9b) had a pKᵢ larger than 7, whereas the only monoalkyl amine that presents a pKᵢ>7 is derivative 4b.

### Agonist or Antagonist

Compounds 4b-9b were subjected to [³⁵S]GTPγS binding experiments to determine their nature as agonists or antagonists and the results are shown in Table 3. The α₂-ARs are G-protein coupled receptors (GPCRs), and as such, when the endogenous ligand binds to the receptor, a change in the conformation of the G-proteins occurs leading to the exchange of GDP by GTP on the α-subunit, promoting their dissociation into α-GTP and βγ subunits, and resulting in transmembrane signalling. A direct evaluation of this G-protein activity can be made by determining the guanine nucleotide exchange using radiolabelled GTP analogues. The [³⁵S]GTPγS binding assay constitutes a functional measure of the interaction of the receptor and the G-protein and is a useful tool to distinguish between agonists (increasing the nucleotide binding), inverse agonists (decreasing the nucleotide binding), and neutral antagonists (not affecting the nucleotide binding) of GPCRs. Experiments were performed in low-affinity receptor conditions for agonists (presence of guanine nucleotides and sodium in the medium), and hence, typical potency values are between two and three logarithmic units lower than affinity values obtained in radioligand receptor binding experiments.

TABLE 3
Affinity for α₂-ARs (pKᵢ), EC₅₀ values and intrinsic activity relative to UK14304
(pKᵢ = 8.85) found for compounds showing a typical agonist dose-response plot

| | | | |
|---|---|---|---|
| Compound | Structure | EC₅₀ (μM)^{†} | Eₘₐₓ (%) |

| | | | |
|---|---|---|---|
| UK14304 | 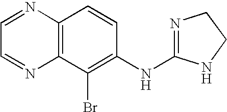 | 11.4 ± 0.3 | 100 |
| 3* | 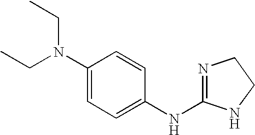 | 2.8 ± 0.1 | 96 |
| 17^{§} | 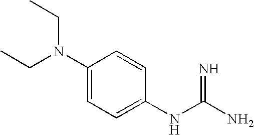 | 38.7 ± 5.0 | 89 |
| 4b | 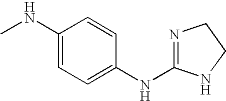 | 61.4 ± 4.9 | 98 |
| 5b | 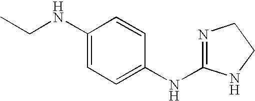 | 59.3 ± 4.9 | 95 |
| 6b | 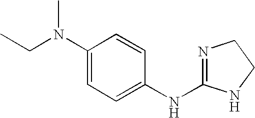 | 1.9 ± 0.1 | 103 |
| 7b | 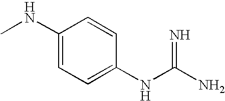 | 82.3 ± 17.0 | 87 |
| 9b | 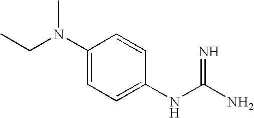 | 16.2 ± 2.3 | 92 |

*Known compound previously prepared by the inventors whose EC₅₀ had been evaluated.
^{§}Known compound previously prepared by the inventors whose EC₅₀ had not been evaluated.
^{†}Cortical membranes from human postmortem brains were incubated at 30° C. for 2 hours in the presence of the different compounds (10⁻¹⁰ M to 10⁻³ M, 8 concentrations).

Compounds 4b, 5b, 6b, 7b, and 9b as well as derivatives 3 and 17 stimulated binding of [³⁵S]GTPγS, showing a typical agonist dose-response plot. Compound 8b alone did not stimulate binding of [³⁵S]GTPγS and was subjected to new [³⁵S]GTPγS experiments and tested against the UK14304.

A rightwards shift of the EC₅₀ for UK14304 when including compound 8b in the assay would confirm its antagonism. Thus, the effect induced in the UK14304 agonist stimulation of [³⁵S]GTPγS binding by the presence of a single concentration (10⁻⁵ M) of derivative 8b was evaluated and is presented in Table 4, along with the effect induced by the known antagonist 1 and the agonist 2.

TABLE 4
EC₅₀ values obtained from the concentration-response
curves for UK14304 stimulation of [³⁵S]GTPγS binding in the absence
or presence of the different compounds (10⁻⁵ M concentration).

| Experiment | EC₅₀ (μM) |
|---|---|
| UK14304 | 11.4 ± 0.3 |
| UK14304 + 1* | 355 ± 18 |
| UK14304 + 2* | 16 ± 2 |
| UK14304 + 8b | 213 ± 18 |

*Compounds previously prepared by the present inventors and evaluated against UK14304; utilised as controls.

Similar to compound 1, addition of derivative 8b produced a remarkable rightwards shift in the EC₅₀ value for the UK14304 (Table 4) indicating that compound 8b behaves as an antagonist in the α₂-ARs in human brain PFC in the experiments carried out in vitro.

In-Vivo Microdialysis Experiments

The antagonistic properties of derivative 8b in-vitro, were substantiated by testing its effect on noradrenergic transmission in vivo using microdialysis experiments. This technique is a widely accepted method for sampling the extracellular fluid of the brain, allowing the study of different neurotransmitters in the extracellular area where the probe is implanted. This technique has been used to investigate the effect of different compounds on NA concentrations in the PFC, an area widely implicated in depression. The increase of NA concentration in the PFC of freely moving rats after drug administration is accepted as a good predictor for antidepressant activity. In this context, many antidepressants, including the α₂-AR antagonist Mirtazapine, are able to increase dialysate levels of NA in the PFC.

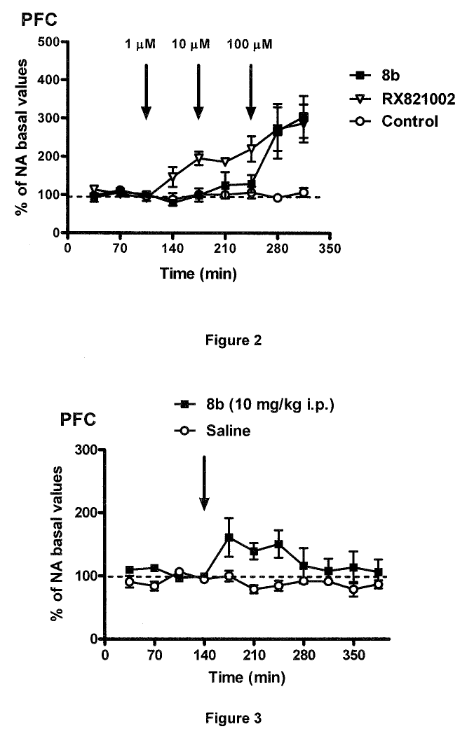

FIG. 2 illustrates that the control of artificial cerebrospinal fluid (aCSF) administration for more than 5 hours did not change NA basal values (F[8,30]=0.58; P=0.77, n=4) in rats. The concentration of the other compounds was progressively increased according to the arrows in the Figure. Compounds 8b and RX821002 were dissolved in aCSF and perfussed via reverse dialysis at the time indicated by the arrows (every 70 minutes). Data correspond to the mean±standard error mean values from 3 to 5 animals for each group, and are expressed as percentages of the corresponding basal values. When derivative 8b was perfussed by reverse dialysis through the probe (1-100 μM), a significant increase in extracellular NA levels was observed (Eₘₐₓ=304±54%, F[1,52]=13.22, P=0.0006 vs control, n=9) at 100 M concentration of compound 8b. The maximal effect was very similar to that obtained from the local administration (1-100 μM) of the well-known α₂-AR antagonist RX821002 (Eₘₐₓ=290±35%, F[1,40]65,32, P<0.0001, n=7).

The effect of derivative 8b on extracellular NA levels by systemic administration was also evaluated as shown in FIG. 3. Control rats were administered with the vehicle (saline). Intraperitoneal administration of compound 8b (10 mg/kg) increased NA extracellular concentration by 161±30% in the PFC. Data are given as mean±standard error mean values from 4-5 separate animals for each group and are expressed as percentages of the corresponding basal values. The arrow represents the time of administration of the compound or vehicle.

This increase was statistically significant when compared with the respective controls (F[1,67]=22.64, P<0.0001, n=9). These results confirm the antagonistic properties shown by compound 8b in vitro and the ability of the compound to cross the blood brain barrier (BBB).

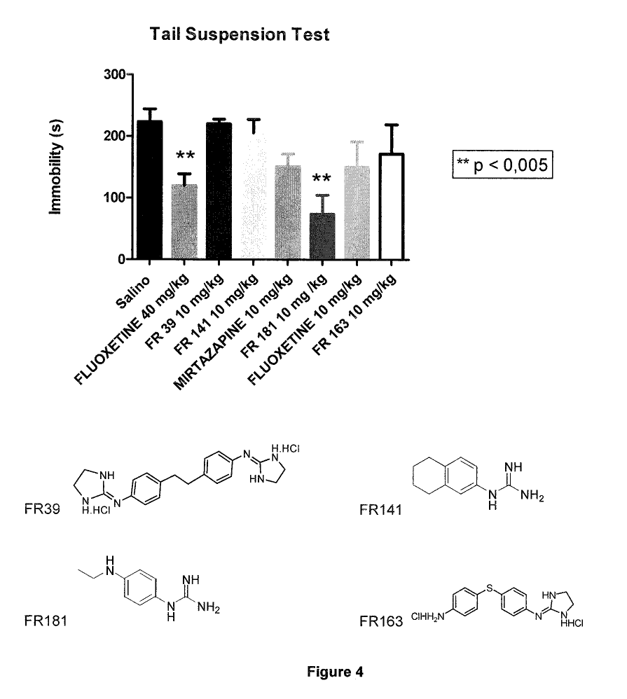

FIG. 4 illustrates the rat tail suspension test for a number of compounds. The tail suspension test has become one of the most widely used models for assessing antidepressant-like activity in mice. The test is based on the fact that animals subjected to the short-term, inescapable stress of being suspended by their tail, will develop an immobile posture. Antidepressant medications reverse the immobility and promote the occurrence of escape-related behaviour.

Notably, compound 8b, or FR181 in FIG. 4, significantly outperforms all other compounds tested. Rats administered with 8b exhibit an immobile posture for the shortest length of time. Of particular note, at a dose of 10 mg/kg compound 8b vastly outperforms one of the most widely prescribed S.S.R.I. antidepressants, fluoxetine, even when fluoxetine is administered at a dose of 40 mg/kg.

Pharmacology: Materials and Methods

Preparation of membranes. Neural membranes (P₂ fractions) were prepared from the PFC of human brains obtained at autopsy in the Instituto Vasco de Medicine Legal, Bilbao, Spain. Postmortem human brain samples of each subject (˜1 g) were homogenized using a Teflon-glass grinder (10 up-and-down strokes at 1500 rpm) in 30 volumes of homogenization buffer (1 mM MgCl₂, and 5 mM Tris-HCl, pH 7.4) supplemented with 0.25 M sucrose. The crude homogenate was centrifuged for 5 min at 1000×g (4° C.) and the supernatant was centrifuged again for 10 min at 40000×g (4° C.). The resultant pellet was washed twice in 20 volumes of homogenization buffer and recentrifuged in similar conditions. Aliquots of 1 mg protein were stored at −70° C. until assay. Protein content was measured according to the method Bradford using BSA as standard, and was similar in the different brain samples.

[³H]RX821002 binding assays. Specific [³H]RX821002 binding was measured in 0.55 ml-aliquots (50 mM Tris HCl, pH 7.5) of the neural membranes which were incubated with [³H]RX821002 (1 nM) for 30 min at 25° C. in the absence or presence of the competing compounds (10⁻¹² M to 10⁻³ M, 10 concentrations). Incubations were terminated by diluting the samples with 5 ml of ice-cold Tris incubation buffer (4° C.), Membrane bound [³H]RX821002 was separated by vacuum filtration through Whatman GFIC glass fibre filters. Then, the filters were rinsed twice with 5 ml of incubation buffer and transferred to minivials containing 3 ml of OptiPhase “HiSafe” II cocktail and counted for radioactivity by liquid scintillation spectrometry. Specific binding was determined and plotted as a function of the compound concentration. Non-specific binding was determined in the presence of adrenaline (10⁻⁵ M).

Analysis of binding data. Analysis of competition experiments to obtain the inhibition constant (Kᵢ) were performed by nonlinear regression using the GraphPad Prism program. All experiments were analysed assuming a one-site model of radioligand binding. Kᵢ values were normalized to pKᵢ values.

[³⁵S]GTPγS binding assays. The incubation buffer for measuring [³⁵S]GTPγS binding to brain membranes contained, in a total volume of 500 μL, 1 mM EGTA, 3 mM MgCl₂, 100 mM NaCl, 50 mM GDP, 50 mM Tris-HCl at pH 7.4 and 0.5 nM [³⁵S]GTPγS. Proteins aliquots were thawed and re-suspended in the same buffer. The incubation was started by addition of the membrane suspension (40 μg of membrane proteins) to the previous mixture and was performed at 30° C. for 120 min with shaking. In order to evaluate the influence of the compounds on [³⁵S]GTPγS binding, 8 concentrations (10⁻¹⁰ to 10⁻³M) of the different compounds were added to the assay. Incubations were terminated by adding 3 mL of ice-cold re-suspension buffer followed by rapid filtration through Whatman GF/C filters pre-soaked in the same buffer. The filters were rinsed twice with 3 mL of ice-cold re-suspension buffer, transferred to vials containing 5 mL. of OptiPhase HiSafe II cocktail (Wallac, UK) and the radioactivity trapped was determined by liquid scintillation spectrometry (Packard 2200CA). The [³⁵S]GTPγS bound was about 7-14% of the total [³⁵S]GTPγS added. Non-specific binding of the radioligand was defined as the remaining [³⁵S]GTPγS binding in the presence of 10 μM unlabelled GTPγS.

Microdialysis experiments Male Sprague-Dawley rats (250-300 g) were implanted with a probe in a stereotaxic apparatus under chloral hydrate anaesthesia (400 mg/kg i.p.). The probe was located in the prefrontal cortex (PFC) according to the co-ordinates of the atlas of Paxinos and Watson (AP (anterior to bregma) +2.8 mm, L (lateral from the mid-sagittal suture) +1 mm, DV (ventral from the dura surface) −5 mm). Experiments were performed 20-24 h after the probe implantation and aCSF (148 mM NaCl, 2.7 mM KCl, 1.2 mM CaCl₂ and 0.85 mM MgCl₂; pH 7.4) was pumped at a flow rate of 1 μl/min (CMA/Microdialysis infusion pump). Drugs, when locally administered, were dissolved in aCSF and applied during 70 min via dialysis probe in increasing concentrations of 1, 10 and 100 μM. Drugs systemically administered were dissolved in saline and injected intraperitoneally. Samples were collected every 35 min and NA concentrations analyzed by HPLC apparatus with amperometric detection (Hewlett-Packard model 1049A) at an oxidizing potential of +650 mV. The mobile phase (12 mM citric acid, 1 mM EDTA, 0.7 mM octylsodio sulphate, pH=5 and 10% methanol) was filtered, degassed (Hewlett-Packard model 1100 degasser) and delivered at a flow rate of 0.2 ml/min by a Hewlett-Packard model 1100 pump. Stationary phase was a column of 150×2.1 mm (Thermo Electron Corporation, U.S.A,). Samples (injection volume 30 μl) were injected and NA analyzed in a run time of 10 min. The mean values of the first three samples before substrate administration were considered as 100% basal value. All measures of extracellular NA concentrations are expressed as percentage of the baseline value±s.e.mean. One-way analysis of variance (ANOVA) for control group or two-way ANOVA between control and each treated group was assessed for statistical analysis. After the experiments, rats were sacrificed with an overdose of chloral hydrate and the brains were dissected to check the correct implantation of the probe.

Drugs. [³H]RX821002 (specific activity 59 Ci/mmol) was obtained from Amersham International, UK. [³⁵S]GTPγS (1250 Ci/mmol) was purchased from DuPont NEN (Brussels, Belgium). Idazoxan HCl was synthesised by Dr. F. Geijo at S. A. Lasa Laboratories, Barcelona, Spain. Clonidine HCl, GDP, GTP, GTPγS, RX821002 HCl, and UK14304 were purchased from Sigma (St. Louis, USA). All other chemicals were of the highest purity commercially available.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A compound comprising,
an aromatic ring, optionally substituted with at least one C₁-C₅ alkyl, selected from the group comprising benzene, thiophene, pyrrole, furan, oxazole, thiazole, pyrazole, pyridine, pyrimidine, pyridazine, and pyrazine;
a single heteroatom, selected from O, N or S, covalently bonded to the aromatic ring, wherein the heteroatom is monoalkylated with a C₁-C₅ alkyl or a C₁-C₅ alkyl chain substituted with at least one of a halogen, hydroxy, thiol, or amine; and
a guanidine, a hydroxyguanidine, 2-aminoimidazole, amidine or isourea moiety optionally substituted with at least one of OH, N-tert-butoxycarbonate, or C₁-C₅ alkyl, covalently bonded:
(a) to the aromatic ring; or
(b) to a C₁-C₅ alkyl or a C₁-C₅ alkyl chain substituted with at least one of a halogen, hydroxy, thiol, or amine, wherein the C₁-C₅ alkyl is covalently bonded to the aromatic ring;
a tautomer thereof, a pharmaceutically acceptable salt thereof, or a hydrate thereof,
with the proviso that when the aromatic ring is benzene having a guanidine or 2-aminoimidazole moiety covalently bound thereto, and the heteroatom is O or S, the heteroatom it is not alkylated with a methyl group.

2. A compound of the general formula (I): 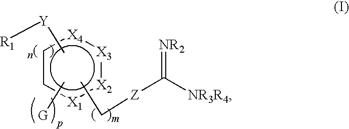
wherein n is 0 or 1;
m is 0 to 5;
p is 0,1 or 2
G is C₁-C₅ alkyl, wherein when p is 2, G₁ and G₂ can be the same or different C₁-C₅ alkyl groups;
Y is N, O or S;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
X₁ to X₄ are the same or different and are selected from the group comprising C, N, S and O;
wherein when n is 0, X₄ is
X₁ is S, O, or N; and
X₂ and X₃ are C or N, with the proviso that X₂ is C when X₁ is S or O; and
further provided that when n is 1, X₁ to X₄ are C or N, such that at least two of X₁ to X₄ are always C;
with the proviso that when X₁ to X₄ are C, n is 1, m is 0, Z and R₂ to R₄ together define a guanidine or 2-aminoimidazole moiety and Y is O or S, R₁ is not methyl,
a tautomer thereof, a pharmaceutically acceptable salt thereof, or a hydrate thereof.

3. A compound according to claim 2 of the general formula (II): 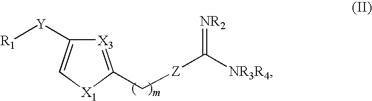
wherein m is 0 to 5;
Y is N, O or S;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
X₁ is N, S or O; and
X₃ is C or N,
a tautomer thereof, a pharmaceutically acceptable salt thereof, or a hydrate thereof.

4. A compound according to claim 2 of the general formula (III): 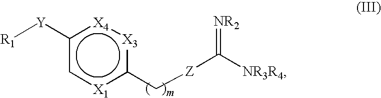
wherein m is 0 to 5;
Y is N, O or S;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
X₁, X₃, and X₄ are C or N, such that at least one of X₁, X₃, and X₄ is always C;
with the proviso that when X₁, X₃, and X₄ are C, m is 0, Z and R₂ to R₄ together define a guanidine or 2-aminoimidazole moiety and Y is 0 or S, R₁ is not methyl,
a tautomer thereof, a pharmaceutically acceptable salt thereof, or a hydrate thereof.

5. A compound according to claim 4 of the general formula (IV): 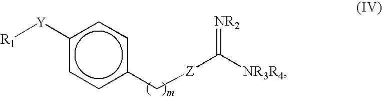
wherein m is 0 to 5;
Y is N, O or S;
R₁ is a C₁-C₅ alkyl or a C₁-C₅ alkyl chain substituted with at least one of a halogen, hydroxy, thiol, or amine;
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring;
Z is O, C or N—R₅;
R₅ is H or C₁-C₅ alkyl;
with proviso that when m is 0, Z and R₂ to R₄ together define a guanidine or 2-aminoimidazole moiety and Y is O or S, R₁ is not methyl,
a tautomer thereof, a pharmaceutically acceptable salt thereof, or a hydrate thereof.

6. A compound according to claim 5 of the general formula (V): 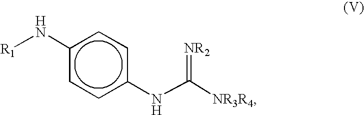
wherein R₁ is a C₂-C₅ alkyl or a C₂-C₅ alkyl substituted with at least one of a halogen, hydroxy, thiol, or amine; and
R₂ to R₄ are the same or different and are selected from the group comprising H, OH, N-tert-butoxycarbonate, or C₁-C₅ alkyl, wherein R₂ and R₄ can together define a bridging ethyl group between both N atoms to form a 5 membered ring,
a tautomer thereof, a pharmaceutically acceptable salt thereof, or a hydrate thereof.

7. A compound according to claim 6 wherein R₁ is a C₂-C₅ alkyl, and R₂ to R₄ are the same or different and are selected from the group comprising H, OH, N-tert-butoxycarbonate, or C₁-C₅ alkyl,
a tautomer thereof, a pharmaceutically acceptable salt thereof, or a hydrate thereof.

8. A compound according to claim 7 wherein R₁ is a C₂-C₅ alkyl, and R₂ to R₄ are H, a tautomer thereof, a pharmaceutically acceptable salt thereof, or a hydrate thereof.

9. A compound according to claim 2 wherein the compound is 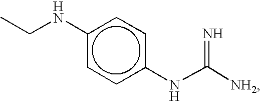
a tautomer thereof, a pharmaceutically acceptable salt thereof, or a hydrate thereof.

10. A compound according to claim 2, a tautomer thereof, a pharmaceutically acceptable salt thereof, or a hydrate thereof, wherein the compound is an alpha2-adrenoceptor antagonist.

11. A pharmaceutical composition comprising a compound according to claim 2, a tautomer thereof, a pharmaceutically acceptable salt thereof, or a hydrate thereof, together with a pharmaceutical acceptable carrier or excipient.

12. A pharmaceutical composition according to claim 11 wherein the compound is 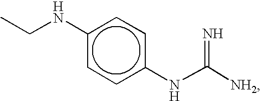
a tautomer thereof, a pharmaceutically acceptable salt thereof, or a hydrate thereof.

13. A method of treating an alpha2-adrenoceptor associated disorder in a patient in need thereof, comprising administering to the patient a pharmaceutically effective amount of a compound according to claim 2, a tautomer thereof, a pharmaceutically acceptable salt thereof, or a hydrate thereof.

14. A method according to claim 13 wherein the alpha2-adrenoceptor associated disorder is selected from at least one of depression or schizophrenia.

15. A method according to claim 14 wherein the alpha2-adrenoceptor associated disorder is depression.

16. An alpha2-adrenoceptor agonist selected from the group comprising: 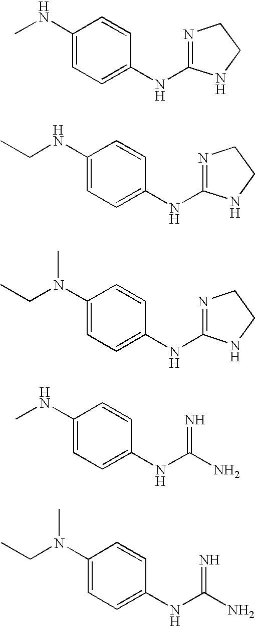
a tautomer thereof, a pharmaceutically acceptable salt thereof, or a hydrate thereof.

17. A method of treating an alpha2-adrenoceptor associated disorder in a patient in need thereof, comprising administering to the patient a pharmaceutically effective amount of an alpha2-adrenoceptor agonist according to claim 16, a tautomer thereof, a pharmaceutically acceptable salt thereof, or a hydrate thereof.

18. A method according to claim 17 wherein the alpha2-adrenoceptor associated disorder may be selected from at least one of analgesia, hypertension or glaucoma.
